(19) United States
(12) Patent Application Publication
(10) Pub. No.: US 2007/0276172 A1
(43) Pub. Date: Nov. 29, 2007

(54) Single step microwave induced process for the preparation of substituted stilbenes and its analogs

(76) Inventors: Arun Kumar Sinha, Palampur (IN); Anuj Sharma, Palampur (IN); Vinod Kumar, Palampur (IN)

Correspondence Address: DLA PIPER US LLP, 4365 EXECUTIVE DRIVE, SUITE 1100, SAN DIEGO, CA, 92121-2133, US

(21) Appl. No.: 11/729,994

(22) Filed: Mar. 28, 2007

(30) Foreign Application Priority Data
Mar. 28, 2006 (IN) 856/DEL/2006

(51) Int. Cl. C07C 2/72

(52) U.S. Cl. 585/428

(57) ABSTRACT

The present invention relates to a single step, microwave induced process for the preparation of substituted stilbenes and its analogs. Particularly, provides a method for the preparation of commercially important 2- or 4-hydroxy substituted stilbenes in one pot utilizing cheaper substrates in the form of 2- or 4-hydroxy substituted arylaldehyde and/or phenylacetic acids as well as regents in the form of base such as collidine, triethylamine, pyridine, piperidine, sodium acetate, ammonium acetate, imidazole, methyl imidazoles and the like and/or acid such as formic acid, acetic acid, propionic acid and the like for a reaction time varying from 1 min-16 hrs depending upon microwave or conventional heating, without using decarboxylating agents with yield varying from 37-66% depending upon the base and/or acid, solvent and substrate used. It is important to mention that the presence of hydroxy substitution at 2- or 4-position of arylaldehyde and/or aryl acetic acid is essential requirements towards formation of stilbenes in one step.

### CROSS REFERENCE TO RELATED APPLICATION

This application is a utility application and claims the benefit under 35 USC § 119(a) of India Application No. 856/DEL/2006 filed Mar. 28, 2006. This disclosure of the prior application is considered part of and is incorporated by reference in the disclosure of this application.

### BACKGROUND OF THE INVENTION

1. Field of the Invention

The present invention relates to a “single step microwave induced process for the preparation of substituted stilbenes and its analogs” wherein, some immensely important compounds are synthesized such as resveratrol (3,4′,5-trihydroxy-(E)-stilbene), pterostilbene (3,5-dimethoxy-4′-hydroxy stilbene) (S. Eddarir, Z. Abdelhadi, C. Rolando, Tetrahedron lett., 2001, 42, 9127); in one pot during condensation of substituted arylaldehyde and substituted aryl acetic acid with at least one hydroxy substituent at 2- or 4-position of either arylaldehyde or aryl acetic acid, under microwave irradiation using base and/or an acid and solvent. Base is selected from a group consisting of collidine, triethylamine, pyridine, piperidine, methyl imidazoles, sodium acetate, ammonium acetate, imidazole, methyl imidazoles and the like. Acid is selected from a group consisting of formic acid, acetic acid, propionic acid and the like. Solvent for the process is selected from a group consisting of ethylacetate, dimethylformamide, ethanediol, diethylene glycol, dimethoxyethylene glycol, dimethyl sulphoxide, ionic liquids and the like. The reaction completes without the use of any decarboxylating agents. The reaction time varies from 1 min to 16 hrs depending upon the base and/or acid, solvent, substrate used and type of microwave, monomode or multimode or conventional heating with yield varying from 37-66%. In addition, we disclose that the presence of hydroxy substitution at 2- or 4-position of arylaldehyde or aryl acetic acids and more importantly employing microwave condition are essential for the enhancement of yield of product stilbenes. The above arylaldehydes and arylacetic acids undergo condensation as well as decarboxylation without use of any decarboxylating agent. The same reaction under conventional method provides the condensation product, substituted aryl acrylic acids (for example α-phenylcinnamic acid) as the major product and stilbenes in low yield as compared to that under microwave irradiation. In the present invention, the formation of substituted stilbenes is the first example from hydroxy substituted arylaldehyde and arylacetic acid in one step without use of decarboxylating agent.

### BACKGROUND INFORMATION

Naturally occurring non-nutritive agents such as flavonoids, phenolic compounds and many others present in plants are believed to have disease preventive properties (S. M. Kau, Oncogenesis, 1997, 8, 47). The clinical relevance of such natural phytochemicals is dependent on extrapolation from epidemiological data and from experiments in animal models of diseases of interest. Substituted stilbenes, is one such class which includes one of the most important therapeutic agents for the prevention of fatal diseases like cancer and heart diseases. For example, resveratrol, a phytoalexin, present in grapes and other fruits (J. Burns, T. Yokota, H. Ashihara, M. E. J. Lean, A. Crozier, J. Agric. Food Chem., 2002, 50, 3337; G. J. Soleas, E. P. Diamandis, D. M. Goldberg, Clin. Biochem., 1997, 30, 91); have been reported to play a role in the prevention of heart diseases associated with red wine consumption because of its properties of platelet aggregation (C. R. Pace-Asciak, S. Hahn, E. P. Diamandis, G. Soleas, D. M. Goldberg, Clin. Chem. Acta., 1995, 235, 207), eicosanoid synthesis alteration (Y. Kimura, H. Okuda, S. Arichi, Biochim. Biophys. Acta., 1985, 834, 275); lipid lowering-activity and lipid peroxidation-inhibition (L. Belguendouz, L. Fremont, M. T. Gozzellino, Biochem. Pharmacol., 1998, 55, 811; E. N Frankel., A. L Waterhouse, J. E. Kinsella, Lancet., 1993, 341, 1103). The low rate of coronary heart diseases in France compared to other Western countries despite the similar risk factors (high animal fat intake, low exercise level, and high rate of smoking) has been called the French paradox. Also that French has the highest consumption of wine than other countries studied, which was later found out to possess resveratrol, a compound inhibiting synthesis of thromboxane in platelets and leukotrienes in neutrophils, and modulate the synthesis and secretion of lipoproteins in animals and human cell lines. Moreover, resveratrol prevented chemical induction of preneoplastic lesion in a mouse mammary gland culture model and could slowdown the growth of skin tumors which had been initiated in mice by a two step carcinogenic stimulus. This effect is proposed to act through the inhibition of cyclo-oxygenase and hydro-peroxide enzymes, by anti-oxidant activity and by inducing differentiation of the cancer cells. It is shown that resveratrol, can easily inhibit dioxin-induced phase I enzymes activity as well as interleukin-I beta production and HIV promoter induction. It can, therefore, protect against a variety of diseases associated with AhR ligand. Hence resveratrol acts as AhR antagonist and thus helps preventing cancer and viral infections such as AIDS (W. K. Bock, Physiol, Biochem. Pharmacol., 1994, 125, 1; J.-F Savouret, M. Antenos, M. Quesne, J. Xu, E. Milgrom, R. F. Casper, J. Boil. Chem., 2001, 276, 3054; M. Poirot, P. De Medina, F. Delarue, J. J. Perie, A. Klaebe, J. C. Faye, Bioorg. Med. Chem., 2000, 8, 2007). This compound is also known to possess anti-inflammatory activities due to down regulation of prosatgladin and prostacyclinsynthesis (Science, 1995, 267, 1782). Resveratrol is also shown to act as an anti-mutagenic compound by inhibiting the cellular events associated with tumor initiation, promotion and progression (Science, 1997, 275, 218). More recently this compound, resveratrol is also shown to act in anti-dandruff formulations (M. Derosa, M. Rossi, U S Patent No. US 2003228269 A1). Similarly, combretastatin A-4, a stilbene, isolated from the African bush willow, Combretum caffrum shows exciting potential as an anti-cancer agent, binding strongly to tubulin and displaying potent and selective toxicity towards tumor vasculature (U.S. Pat. No. 4996; Brit. J. Cancer, 1999, 81, 1318; Brit. J. Cancer, 1995, 71, 705). Combretastatin A-4 is able to elicit irreversible vascular shutdown within solid tumors, leaving normal vasculature intact (E. Hamel, C. M. Lin, Biochem. Pharmacol., 1983, 32, 3863; D. J. Chaplin, G. R. Pettit, C. S. Parkins, S. A. Hill, Brit. J. Cancer, 1996, 74, S86; G. G. Dark, S. A. Hill, V. E. Prise, G. M. Tozer, G. R. Pettit, D. J. Chaplin, Cancer Res., 1997, 57, 1829). Moreover, pterostilbene, reported to be the only stilbene present in the genus Pterocarpus is also found in Vitis vinifera leaves. It was found to be one of the most active components in the extract of heartwood of P. marsupium, used in the Ayurvedic medicines for the treatment of diabetes. It was reported to act as hypoglycemic compound which significantly lowered the plasma glucose levels in the hyperglycemic rats. The compound was also reported as an anti-oxidant. Similarly, there are other stilbenoids which exhibit a wide range of therapeutic properties. For instance, trimethyl ether of resveratrol shows more activity against several human cancer cells than resveratrol (G. R. Pettit, M. P. Grealish, M. K. Jung., E. Hamel, R. K. Pettit, J. C. Chapuis, J. M. Schmidt, J. Med. Chem., 2002, 45, 2534 and references cited therein). Similarly, 3,5-dimethoxy-(2′-thienyl)-stilbene and 2′,3,4′,5-tetramethoxy stilbene are reported to be potent CYP1B1 inhibitors valuable for the development of the chemo preventive therapy against cancer (S. Kim, H. Ko, J. E. Park, S. Jung, S. K. Lee, Y.-J. Chun, J. Med. Chem., 2002, 45, 160). Similarly, other hydroxy substituted stilbenes also have profound applications in the medicinal field (A. M. Rimando, M. Cuendet, C. Desmarchelier, R. G. Mehta, J. M. Pezzuto, S. O. Duke, J. Agric. Food Chem., 2002, 50, 3453). In the pretext of above discussion, 2- or 4-hydroxy substituted stilbenes and their analogues can unhesitatingly be counted as greatly valued to humankind. Some of them, as already explained, are found in nature, however, the limited percentage of these hydroxy substituted stilbenes in plant kingdom is not sufficient to fulfill the world demand. So, the bulk amount of 2- or 4-hydroxy stilbene could be prepared synthetically. A number of chemical methods are reported in literature for the preparation of hydroxy substituted stilbenes and their analogues which involve Wittig type and modified Julia olefination, reaction of benzyllithium with benzaldehydes followed by dehydration, Perkins reaction, cross metathesis of styrenes, Suzuki reaction with B-halostyrenes, decarbonylative. Heck reaction between acid chloride and styrene, Heck arylation-desilylation of vinylsilane followed by Heck arylation of styrenes formed in situ and Palladium catalysed arylation of styrenes with halobenzene (G. R. Pettit, M. P. Grealish, M. K. Jung, E. Hamel, R. K. Pettit, J.-C. Chapuis, J. M. Schmidt, J. Med. Chem., 2002, 45, 2534; M. Roberti, D. Pizzirani, D. Simony, R. Rondanin, R. Baruchello, C. Bonora, F. Buscemi, S. Grimaudo, M. Tolomeo, J. Med. Chem., 2003,46, 3546; H. Meier, U. Dullweber, Tetrahedron Lett., 1996, 37, 1191; J. Yu, M. J. Gaunt, J. B. Spencer, J. Org. Chem., 2002, 67,4627; D. A. Alonso, C. Nájera, M. Varea, Tetrahedron Lett., 2004, 45, 573; E. Alonso, D. J. Ramón, M. Yus, J. Org. Chem., 1997, 62, 47; G. Solladié, Y. Pasturel-Jacopé, J. Maignun, Tetrahedron, 2003, 59, 3315; S. Chang, Y. Na, H. J. Shin, E. Choi, L. S. Jeong, Tetrahedron Lett., 2002, 43, 7445; S. Eddarir, Z. Abdelhadi, C. Rolando, Tetrahedron Lett., 2001, 42, 9127; M. B. Andrus, J. Liu, E. L. Meredith, E. Nartey, Tetrahedron Lett., 2003,44, 4819; T. Jeffery, B. Ferber, Tetrahedron Lett., 2003,44, 193; N. F. Thomas, K. C. Lee, T. Paraidathathu, J. F. F. Weber, K. Awing, Tetrahedron Lett., 2002, 43, 3151).

The following prior art references are disclosed as below:

Journal of Org. Chem., 2001, 66, 8135, discloses a method for the synthesis of combretastatin A-4 (both cis and trans isomeric forms) through Wittig method and Perkin condensation method.

Natural Product Research., 2006, 20, 247, discloses a method for the improve synthesis of resveratrol through two step process Wittig reaction and Heck coupling.

Synthesis, 2006, 273, discloses a method for the synthesis of biologically important trans-stilbenes via Ru-catalyzed cross metathesis.

J. Med. Chem., 2005, 48, 6783, discloses a method for the synthesis resveratrol analogue with high ceramide-mediated proapoptotic activity on human breast cancer cells.

Molecules, 2004, 9, 658, discloses a method for synthesis of stilbenes via the Knoevenagel condensation.

Carbohydrate Research., 1997, 301, 95, discloses a method for the synthesis of Various hydroxy stilbenes and their glycosides through Wittig reaction.

Bioorg. Med. Chem. Lett. 1998, 8, 1997, discloses a method for the asymmetric synthesis of antimitotic combretadioxolone with potent anti-tumor activity.

Tetrahedron, 2004, 60, 5563, discloses a method for the synthesis of resveratrol and their analogues Heck reaction in organic and aqueous solvents.

J. Med. Chem., 2002, 45, 2534, discloses a method for the synthesis of hydroxy stilbenes and benzophenones through Wittig reaction.

U.S. Pat. No. 20040147788 A1 discloses a method for the synthesis of stilbene derivatives through Wittig reaction.

U.S. Pat. No. 20040015020 A1 discloses a method for the synthesis of E-isomer of stilbene through halide assisted conversion of corresponding Z-isomer.

J. Med. Chem., 2003,46, 3546, discloses a method for the synthesis of resveratrol and their analogues through addition of aromatic aldehydes and appropriate ylide.

Journal of Org. Chem., 1961, 26, 5243, discloses a method for the synthesis of stilbene and heterocyclic stilbene analogs.

Journal of Chem. Soc., 1954, 3596, discloses a method for the synthesis of stilbenes through dehydrogenation from diarylethanes.

U.S. Pat. No. 6,048,903 discloses a method for the synthesis of E-resveratrol by Wittig reaction comprising benzyltriaryl phosphonium salt and anisaldehyde in the presence of n-butyl lithium.

Organic Synthesis Collective Volume I, 1941, 441-442 as well as Volume IV, 1963, 731-734, disclose a method for the preparation of styrenes by decarboxylation of cinnamic acids with quinoline in the presence of copper powder at 200-300° C.

Some of other typical prior art references include U.S. Pat. Nos. 6,844,471, 6,552,213, 20040152629 A1; 6,361,815; 5,569,786; European Pat. Nos. EP 0331983; Tetrahedron lett., 1980, 21, 2073; Synthesis, 1977, 58, J. Chem. Soc. Perkin Trans. I, 1974, 961; J. Chem. Soc., 1963, 2875; Chem. Pharm. Bull., 1992,40, 1130. Although, the above mentioned methods have been proven to be useful, they suffer from one or more process deficiencies. For example, in most of the reported cases, it requires multi-step processes to obtain stilbenes and some others resort to sub-ambient temperatures, which of course, involves some considerable process control and leads to reaction mixtures.

It therefore, becomes an object of the invention to provide rapid and economical process for the preparation of 2- or 4-hydroxy substituted stilbenes from cheaper and commercially available substituted arylaldehyde and arylacetic acids as well as to eliminate the disadvantages associated with the above patents and papers.

### OBJECTIVES OF THE INVENTION

The main object of the present invention is to provide a single step microwave induced process for the preparation of substituted stilbenes and its analogues.

Another object of the present invention is to prepare high valued medicinally important 2- or 4-hydroxy substituted stilbenes from condensation of substituted arylaldehyde and substituted aryl acetic acid with at least one hydroxy substituent at 2- or 4-position of either arylaldehyde or aryl acetic acid.

Yet another object of the present invention is to provide a process for the preparation of 2- or 4-hydroxy substituted stilbenes under microwave or conventional conditions.

Yet another object of the present invention is to provide a process for the preparation of 2- or 4-hydroxy substituted stilbenes where both condensation and decarboxylation occurred in one step without addition of decarboxylating agent.

Yet another object of the invention is to provide a process to prepare 2- or 4-hydroxy substituted stilbenes in good yield.

Yet another object of the invention is to provide a simple process for the preparation of 2- or 4-hydroxy substituted stilbenes in high purity with minimum side products.

Yet another object of the invention is to provide a process in which some of condensing organic acids and organic bases such as piperidine and acetic acid are FEMA GRAS approved which makes our process even safer and eco-friendly.

Yet another object of the invention is to provide a process wherein the ionic liquids used as solvents are recyclable.

Still another object of the invention is to provide a process which utilizes less or non-hazardous chemicals.

Still another object of the invention is to provide a process which requires cheaper chemical reagents.

Yet another object of the invention is to develop industrially viable and economical process towards formation of high valued 2- or 4-hydroxy substituted stilbenes.

Yet another object of the invention is to develop a process wherein the substrate used should have at least one hydroxy substitution at 2- or 4-position of aryl aldehydes or aryl acetic acids.

### SUMMARY OF THE INVENTION

Accordingly, the present invention provides a process for the preparation of commercially important pharmacologically active 2- or 4-hydroxy substituted stilbenes such as resveratrol, pterostilbene, and many others in one pot under microwave irradiation from the condensation of substituted arylaldehydes and substituted aryl acetic acids with at least one hydroxy substituent at 2- or 4-position of either arylaldehyde or aryl acetic acid in the presence of a base and/or an acid and solvent. Base is selected from a group consisting of collidine, triethylamine, pyridine, piperidine, sodium acetate, ammonium acetate, imidazole, methyl imidazoles and the like. Acid is selected from a group consisting of formic acid, acetic acid, propionic acid and the like. Solvent for the process is selected from a group consisting of ethylacetate, dimethylformamide, ethanediol, diethylene glycol, dimethoxyethylene glycol, dimethyl sulphoxide, ionic liquids and the like. The final product i.e. 2- or 4-hydroxy substituted stilbene is obtained in good to moderate yield varying from 37-66% within 1 min-16 hrs. It is worthwhile to mention that this microwave-assisted unique process is in fact an unexpected result of two individual steps (i.e. condensation and decarboxylation) observed for the first time during condensation of substituted arylaldehyde and substituted aryl acetic acid with at least one hydroxy substituent at 2- or 4-position of either arylaldehyde or aryl acetic acid in one step without addition of decarboxylating agent. It is also important to note that conducting the above reaction by conventional method instead of microwave provides aryl acrylic acid as a major product and low yield of stilbene, even when 2- or 4-hydroxy substituted arylaldehyde and aryl acetic acid are taken as starting materials.

### BRIEF DESCRIPTION OF THE DRAWINGS

FIG. 1 is ¹H NMR (300 MHz) spectrum of 4-hydroxy-3,4′-dimethoxy stilbene in CDCl₃ (Example I).

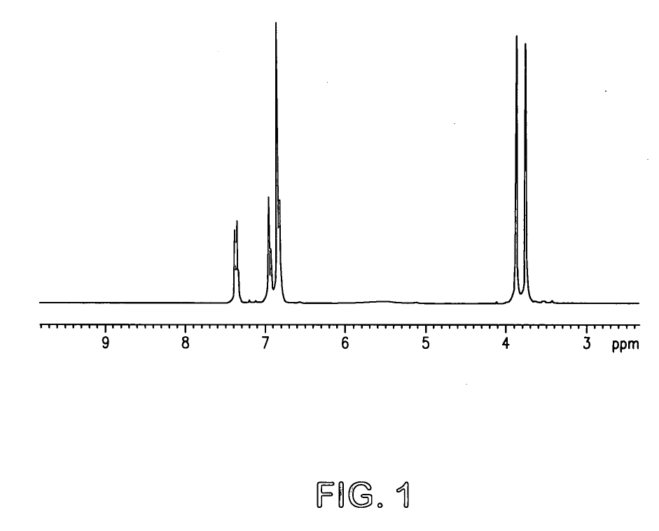

FIG. 2 is ¹³C NMR (75.4 MHz) spectrum of 4-hydroxy-3,4′-dimethoxy stilbene in CDCl₃ (Example I).

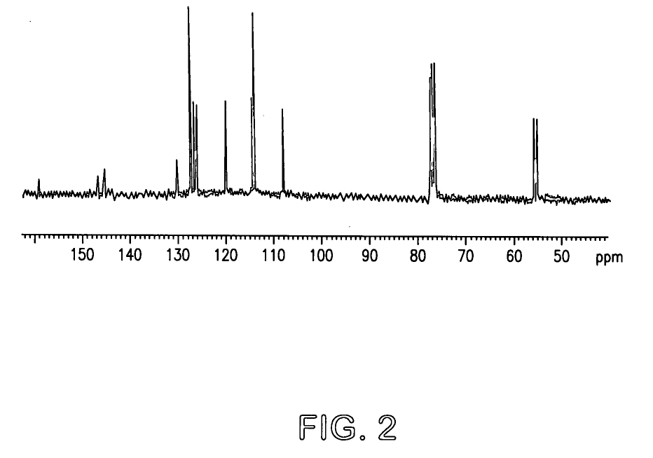

FIG. 3 is HRMS spectrum of 4-hydroxy-3,4′-dimethoxy stilbene. (Example I).

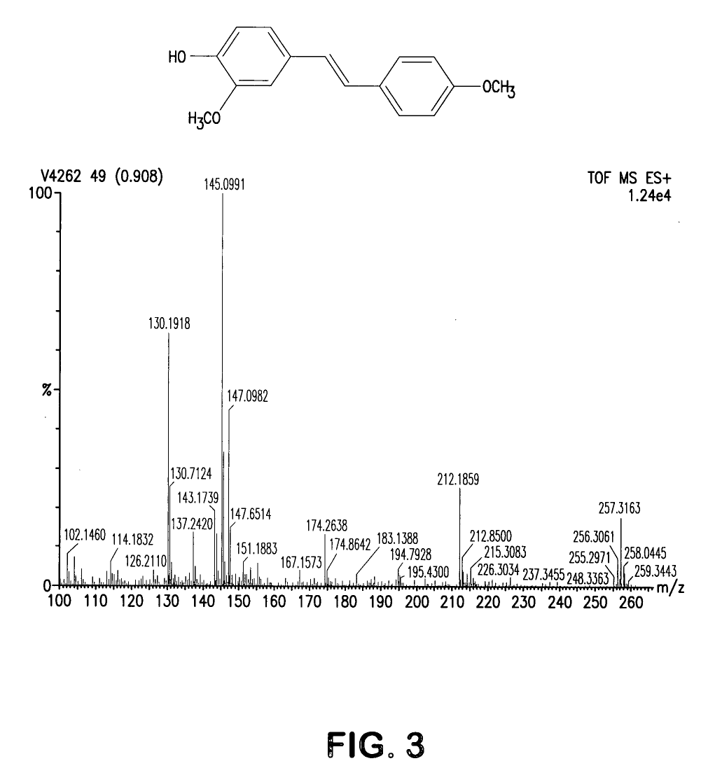

FIG. 4 is ¹H NMR (300 MHz) spectrum of 4-hydroxy-3′,4′-dimethoxy stilbene in MeOD. (Example II).

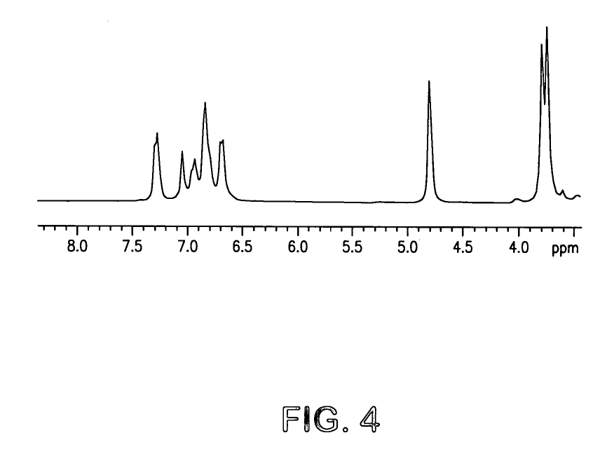

FIG. 5 is ¹³C NMR (75.4 MHz) spectrum of 4-hydroxy-3′,4′-dimethoxy stilbene in MeOD. (Example II).

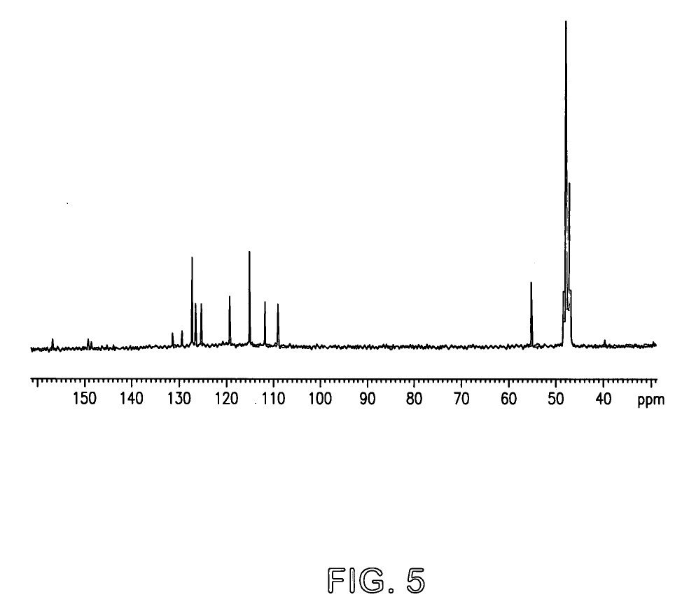

FIG. 6 is HRMS spectrum of 4-hydroxy-3,4′-dimethoxy stilbene. (Example II).

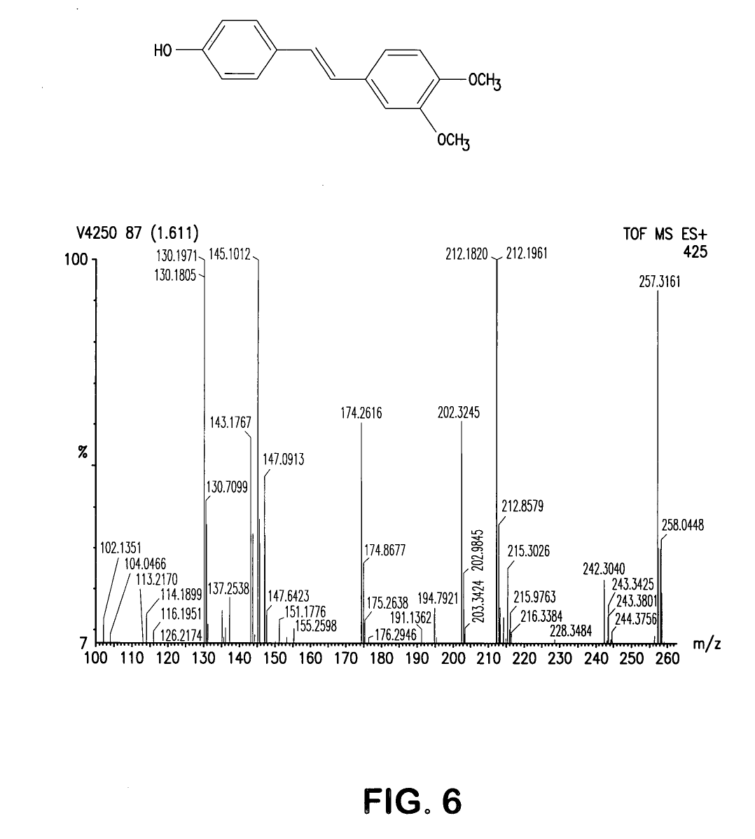

FIG. 7 is ¹H NMR (300 MHz) spectrum of 4-hydroxy-3′,5′-dimethoxy stilbene (petrostilbene) in CDCl₃. (Example III).

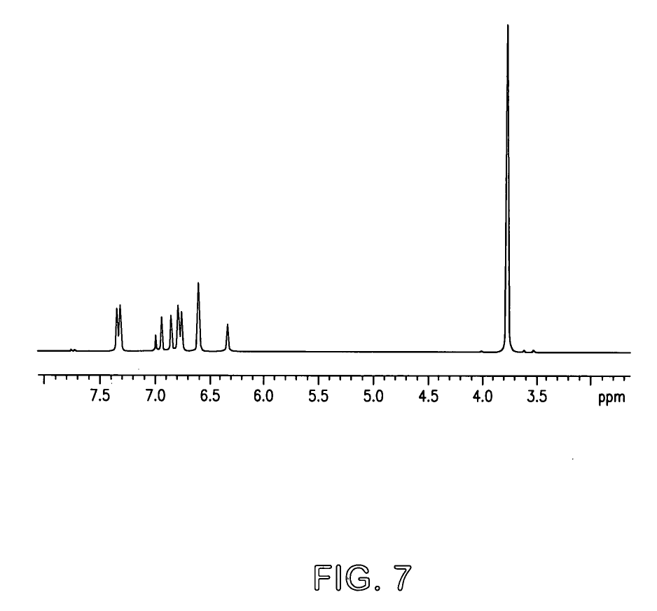

FIG. 8 is ¹³C NMR (75.4 MHz) spectrum of 4-hydroxy-3′,5′-dimethoxy stilbene (petrostilbene) in CDCl₃. (Example III).

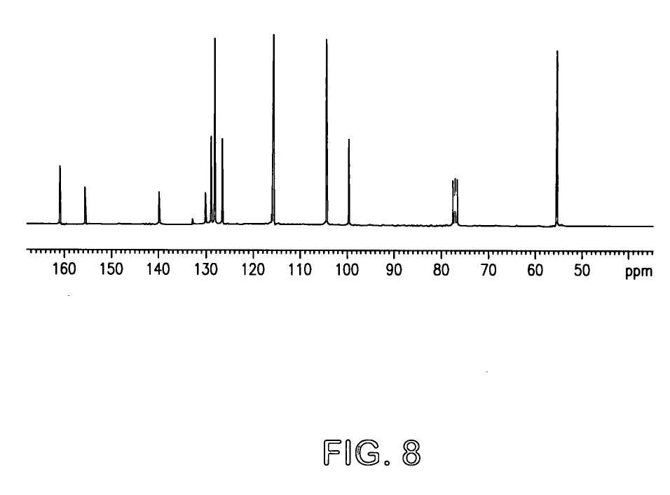

### DETAILED DESCRIPTION OF THE INVENTION

In the pretext of above discussion, 2- or 4-hydroxy substituted stilbenes and their analogues can unhesitatingly be counted as greatly valued to humankind. However, most of the above mentioned methods have various limitations, for example, low yield, expensive reagents and formation of unwanted side products. It, therefore, becomes an object of the invention to provide rapid and economical process for the preparation of 2- or 4-hydroxy substituted stilbenes from cheaper and commercially available substituted arylaldehyde and arylacetic acids as well as to eliminate the disadvantages associated with the above patents and papers. It is worthwhile to mention that microwave-assisted (A. K. Bose, B. K. Banik, N. Lavlinskaia, M. Jayaraman, M. S. Manhas, Chemtech, 1997, 27, 18; M. Larhed, Hallberg, Drug Discovery Today, 2001, 6(8), 406) chemical transformation is a new emerging technique which is generally known for ecofriendly, rapid and high yielding process, however, such an effect of microwave is unique in the above invention in a way that both condensation and decarboxylation have occurred simultaneously without addition of decarboxylating agent.

Keeping in view the above problems, we disclose a unique and novel process to prepare 2- or 4-hydroxystilbenes and their analogous (Examples I-V) in one step from 2- or 4-hydroxy substituted arylaldehyde and/or arylacetic acids in the presence of a base, an acid and a solvent. In fact, we have already observed that while trying to emulate Knoevenagel Doebner condensation (B. S. Fumiss, A. J. Hannaford, V. Rogers, P. W .G. Smith, A. R. Tatchell, In: Vogel's Textbook of Practical Organic Chemistry, Fourth Edn. (ELBS, UK), 1978, 802), reaction under microwave (A. K. Bose, B. K. Banik, N. Lavlinskaia, M. Jayaraman, M. S. Manhas, Chemtech, 1997, 27, 18; M. Larhed, Hallberg, Drug Discovery Today, 2001, 6(8), 406; C. Kuang, H. Senboku, M. Tokuda, Tetrahedron, 2002, 58, 1491; N. Kuhnert, Angew. Chem. Int. Ed., 2002, 41, 1863) 4-hydroxy substituted benzaldehydes produce aryl styrenes instead of the expected cinnamic acids (A. K Sinha., B. P Joshi., A Sharma., US Patent Ser. No. 06/989,467, 2006). This prompted us to extend the same methodology for the synthesis of stilbenes. Hence, in the beginning, we decided to react 4-hydroxy-3-methoxy benzaldehyde with 4-methoxy phenylacetic acid in the Knoevenagel Doebner manner utilizing an acid and a base under microwave irradiation for 1-5 minutes as disclosed in the earlier patent filed by us. However, there was no occurrence of the reaction which demanded some modification in the above procedure. The amount of phenyl acetic acid was increased, various combinations of acids, bases and solvents were used to optimize the reaction conditions which produced the product in moderate to good yield. The compound was analysed on the basis of its spectral data (NMR, Mass etc.) and was found to be matching with the reported values. The same method was applied on other 2- or 4-hydroxy substituted benzaldehydes and upon condensation with phenylacetic acid it successfully provided the required stilbenes wherein, both condensation as well as decarboxylation steps got completed in a single step. Similar results were obtained when 2- or 4-hydroxy substituted phenyl acetic acid is condensed with the substituted benzaldehydes. In this case also condensation and decarboxylation occur in a single step. The reaction was also performed under refluxing on heating mental instead of microwave conditions (Example V) and stilbene was obtained, but in low yield along with α-phenyl cinnamic acid. This clearly shows that microwave enhances the yield of product along with the reduction in reaction time.

In conclusion, our invention discloses a simple and economical process for preparing 2- or 4-hydroxy substituted stilbenes starting from relatively cheaper and economical material substituted arylaldehyde and substituted aryl acetic acid with at least one hydroxy substituent at 2- or 4-position of either arylaldehyde or aryl acetic acid in the presence of a base, an acid and solvent under microwave or conventional conditions which avoid the use of any decarboxylating agent.

Accordingly, the present invention provides a single step microwave induced process for the preparation of substituted stilbenes and its analogs of general formula I wherein, at least one substituent being OH amongst R₁, R₃, R₅, R₆, R₈, R₁₀, and rest of substituents amongst R₁ to R₁₀, being H or OH or OCH₃ or CH₃COO or halogen or nitro or combinations thereof, the said process comprising steps of: 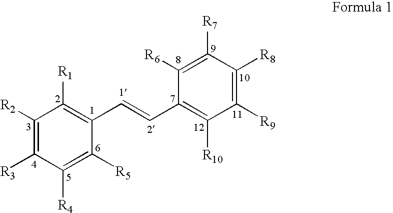
a) reaction of substituted arylaldehyde and substituted aryl acetic acid with at least one hydroxy substituent at 2- or 4-position of either arylaldehyde or aryl acetic acid in the presence of a base and/or an acid and a solvent by refluxing under conventional or microwave irradiation for 1 min-16 hrs,
b) transferring the reaction mixture of step (a) and washing the residue with an organic solvent,
c) washing the organic solution of step (b) with aqueous sodium bicarbonate, brine and water,
d) drying the organic layer of step (c) over anhydrous sodium sulphate, filtering and evaporating to dryness to completely remove the solvent to obtain a residue,
e) purifying the residue of step (d) by column chromatography or recrystallization to obtain the required substituted 2- or 4-hydroxy stilbenes of general formula (I).

In another embodiment of the present invention, wherein the developed process is used for the preparation of medicinally important 2- or 4-hydroxy substituted stilbenes from condensation of substituted arylaldehyde and substituted aryl acetic acid with at least one hydroxy substituent at 2- or 4-position of either arylaldehyde or aryl acetic acid.

In another embodiment of the present invention, wherein the process for the preparation of 2- or 4-hydroxy substituted stilbenes occurred in one step without any addition of decarboxylating agent.

In another embodiment of the present invention, wherein the presence of 2- or 4-hydroxy substituent either at arylaldehyde or arylacetic acids or both is must for condensation-decarboxylation to occur in one step.

In another embodiment of the present invention, wherein the product stilbene is formed both under microwave irradiation as well as conventional heating.

In another embodiment of the present invention, wherein the base is selected from a group consisting of collidine, triethylamine, pyridine, piperidine, sodium acetate, ammonium acetate, imidazole, methyl imidazoles and the like.

In another embodiment of the present invention, wherein acid is selected from a group consisting of formic acid, acetic acid, propionic acid and the like.

In another embodiment of the present invention, wherein solvent is selected from a group consisting of ethylacetate, dimethylformamide, ethanediol, diethylene glycol, dimethoxyethylene glycol, dimethyl sulphoxide, ionic liquids and the like.

In another embodiment of the present invention, wherein the base serve the dual purpose both of base as well as of solvent.

In yet another embodiment of the present invention, wherein developed process is applied equally successfully on aromatic ring in arylaldehydes and arylacetic acids other than benzaldehydes and phenylacetic acids such as naphthyl, phenanthryl, pyridyl, indyl, furyl, thiazolyl ring and the like.

In another embodiment of the present invention, wherein microwave enhances the yield of product stilbenes as compared to conventional method.

In another embodiment of the present invention, wherein the developed process is found equally workable in monomode and multimode microwave.

In another embodiment of the present invention, the microwave irradiation frequency used is in the range of 900 to 3000 MHz more preferably 2450 to 2455 MHz.

In yet another embodiment of the present invention, wherein the reaction is performed in a monomode microwave organic synthesizer operated at 50 W-300 W power level with 100-250° C. for 1-20 min.

In another embodiment of the present invention, wherein the temperature attained in case of monomode microwave is ranging from 100-250° C. preferably 120-190° C.

In another embodiment of the present invention, wherein the reaction is successfully performed in a domestic microwave oven operated at 700 W-1500 W power level for 1 min-30 min.

In another embodiment of the present invention, wherein the product is formed by refluxing substrates for 2-16 hrs preferably 2-6 hrs.

In another embodiment of the present invention, wherein the mole ratio between substituted arylaldehyde and arylacetic acids is ranging from 1:1 to 1:4 moles.

In another embodiment of the present invention, wherein the mole ratio between the substituted arylaldehyde and base is ranging from 1:1 to 1:10 moles preferably 1:3 moles.

Yet another embodiment of the present invention, wherein the mole ratio between the substituted arylaldehyde and acid is ranging from 1:1 to 1:20 moles preferably 1:10 moles.

In yet another embodiment of the present invention wherein developed process provides 2- or 4-hydroxy substituted stilbenes in high purity with no or minimum side products.

In yet another embodiment of the present invention, wherein developed process can be used for the preparation of large number of substituted stilbenes, ethenes and analogs by taking different substrate.

### EXAMPLES

The invention is further illustrated with the help of the following examples and should not be construed to limit the scope of the present invention.

The starting material substituted arylaldehyde including 2- or 4-hydroxy arylaldehyde such as vanillin, 2- or 4-hydroxybenzaldehyde or the like and substituted aryl acetic acids can be obtained from commercial sources. Discover CEM synthesizer (300 W) monomode microwave and Kenstar multimode microwave oven (2450 MHz, 1200 Watts) were used for the reactions.

### Example I

### Synthesis of 4-Hydroxy-3,4′-Dimethoxy Stilbene Or 1-(4′-Hydroxy-3′-Methoxy)Phenyl-2-(4″-Methoxy)Phenylethene

### From Formula I Where R₃=OH, R₂ And R₈=OCH₃, R₁, R₄, R₅, R₆, R₇, R₉, R₁₀=H

A mixture of 4-hydroxy-3-methoxybenzaldehyde (0.0164 mol), 4-methoxyphenylacetic acid (0.0181 mol), piperidine (3 ml) and acetic acid (4 ml) were taken in a 100 ml round bottom flask fitted with a condenser. The flask was shaken well and placed inside the microwave oven (monomode, 200 W, 140° C.) and irradiated for 10 minutes in parts. The cooled mixture was poured into ice-cold water and extracted with ethyl acetate. The organic layer was washed with water, brine and then organic layer dried over sodium sulphate. The solvent was evaporated under reduced pressure to obtain liquid which was purified on silica gel by column chromatography using a mixture of hexane and ethyl acetate (9:1 to 6:4), provided a crystalline white solid; 55% yield (m.p. 163-166° C.); ¹H NMR (CDCl₃) δ 7.37 (2H, d, J=8.48 Hz), 6.95 (2H, d, J=9.28 Hz), 6.84 (5H, m), 3.88 (3H, s), 3.76 (3H, s); ¹³C NMR (CDCl₃) δ 159.0, 146.7, 145.3, 130.4, 127.4, 126.6, 126.1, 120.1, 114.5, 114.1,108.0, 55.9 and 55.3.

### Example II

### Synthesis of 4-Hydroxy-3′,4′-Dimethoxy Stilbene

### From Formula I Where R₃=OH, R₇=R₈ =OMe, R₁, R₂, R₄, R₅, R₆, R₉, R₁₀=H

A mixture of 3,4-dimethoxybenzaldehyde (0.0151 mol), 4-hydroxyphenylacetic acid (0.0604 mol), methyl imidazole (2 ml) and formic acid (4 ml) were taken in a 250 ml Erlenmeyer flask fitted with loose funnel at the top. The flask was shaken well and placed inside the microwave oven (multimode) and irradiated (900 W) for 12 minutes in parts. After completion the reaction was worked up as in example I and provided a white solid; 56% yield (m.p. 180-182° C.) ¹H NMR (MeOD) δ 7.27 (2H, m), 7.04 (2H, d, J=9.28 Hz), 6.94 (5H, m), 6.84 (5H, m), 6.70 (1H, s), 4.80 (solvent peak), 3.79 (3H, s), 3.75 (3H, s); ¹³C NMR (MeOD) δ 156.7, 149.2, 148.5, 131.4, 129.3, 127.2, 126.4, 125.2, 119.2, 115.0, 11.6, 109.0 and 55.0.

### Example III

### Synthesis of 4-Hydroxy-3′,5′-Dimethoxy Stilbene (Pterostilbene)

### From Formula I Where R₃=OH, R₇ And R₉=OCH₃, R₁, R₂, R₄, R₅, R₆, R₈, R₁₀=H

A mixture of 4-hydroxybenzaldehyde (0.0205 mol), 3,5-dimethoxyphenylacetic acid (0.041 mol), piperidine (3 ml) and ethylene glycol (2 ml) were taken in a 100 ml round bottom flask fitted with a condenser. The flask was shaken well and placed inside the microwave oven and irradiated (monomode, 150 W, 180° C.) for 5-8 minutes in parts. After completion the reaction was worked up as in example I and provided a white solid; 66% yield (m.p. 83-86° C.) ¹H NMR (CDCl₃) δ 7.35 (2H, d, J=8.48 Hz), 6.94 (1H, d, J=16.55 Hz), 6.85 (1H, d, J=16.55 Hz), 6.79 (2H, d, J=8.48 Hz), 6.61 (2H, d, J=1.61 Hz), 6.34 (1H, s), 3.77 (3H, s); ¹³C NMR (CDCl₃) δ 160.9, 155.5, 139.7, 130.0, 128.8, 128.1, 126.5, 115.7, 104.5, 99.7 and 55.4.

### Example IV

### Synthesis of 4,4′-Dihydroxy-3-Methoxy Stilbene

### From Formula I Where R₃ And R₈=OH, R₂=OCH₃, R₁, R₄, R₅, R₆, R₇, R₉, R₁₀=H

A mixture of 4-hydroxy-3-methoxybenzaldehyde (0.0164 mol), 4-hydroxyphenylacetic acid (0.041 mol), collidine (2 ml) and ionic liquid (1 g, 1-butyl-3-methylimidazolium chloride) were taken in a 100 ml round bottom flask fitted with a condenser. The flask was shaken well and placed inside the microwave oven and irradiated (monomode, 200 W, 130° C.) for 10-14 minutes in parts. After completion the reaction was worked up as in example I and provided viscous liquid; 52% yield; ¹H NMR (CDCl₃) δ 7.34 (2H, d, J=9.28 Hz), 7.12 (1H, s), 6.97 (3H, m), 6.77 (3H, m), 3.81 (3H, s); ¹³C NMR (CDCl₃) δ 156.8, 147.6, 146.2, 130.0, 129.5, 127.4, 126.9, 125.7, 119.8, 115.5, 115.0, 108.9and55.3.

### Example V

### Synthesis of 4′-Chloro-4-Hydroxy-3-Methoxystilene

### From Formula I Where R₃=OH, R₂=OCH₃ And R₈=Cl, R₁, R₄, R₅, R₆, R₇, R₉, R₁₀=H

A mixture of 4-hydroxy-3-methoxybenzaldehyde (0.0164 mol), 4-chlorophenylacetic acid (0.0181 mol), pyridine (2ml), and methylimidazole (3 ml) were taken in a 100 ml round bottom flask fitted with a condenser. The flask was shaken well and placed inside the microwave oven and irradiated (monomode, 220 W, 140° C.) for 10-16 minutes in parts. After completion the reaction was worked up as in example I and provided a white solid; 62% yield (m.p. 121-124° C.); ¹H NMR (CDCl₃) δ 7.35 (2H, d, J=8.07 Hz), 7.25 (2H, d, J=8.07Hz), 6.96 (3H, m), 6.87 (2H, m), 5.72 (1H, s), 3.87 (3H, s); ¹³C NMR (CDCl₃) δ 146.8, 145.8, 136.1, 132.7, 129.6, 129.2, 128.8, 127.4, 125.1, 120.6, 114.6, 108.3 and 55.9.

### Example VI

### Synthesis of 2-Hydroxy-3,4′-Dimethoxy Stilbene

### From Formula I Where R₁=OH, R₂ And R₈=OCH₃, R₃, R₄, R₅, R₆, R₇, R₉, R₁₀=H

A mixture of 2-hydroxy-3-methoxybenzaldehyde (0.0164 mol), 4-methoxyphenylacetic acid (0.0181 mol), ammonium acetate (2-4g) and diethylene glycol (3 ml) were taken in a 100 ml round bottom flask fitted with a condenser. The flask was shaken well and placed inside the microwave oven and irradiated (monomode, 250 W, 150° C.) for 6-9 minutes in parts. After completion the reaction was worked up as in example I and provided a solid; 48% yield; ¹H NMR (CDCl₃) δ 7.45 (2H, d, J=8.07 Hz), 7.29 (1H, d, J=16.15Hz), 7.13 (2H, d, J=8.07 Hz), 6.84 (4H, m), 5.94 (1H, s), 3.83 (3H, s), 3.76 (3H, s); ¹³C NMR (CDCl₃) δ 159.1, 146.7, 143.2, 130.7, 128.9, 127.8, 124.0, 120.1, 119.5, 118.6, 114.0, 109.1, 56.3 and 55.3.

### Example VII

### Synthesis of 3′,4-Dihydroxy-3,5-Dimethoxy Stilbene

### From Formula I Where R₃ And R₇=OH, R₂ And R4=OCH₃, R₁, R₅, R₆, R₈, R₉, R₁₀=H

A mixture of 4-hydroxy-3,5-dimethoxybenzaldehyde (0.0137 mol), 3-hydroxyphenylacetic acid (0.0151 mol), sodium acetate (4g) and polyethylene glycol (2-3 ml) were taken in a 100 ml round bottom flask fitted with a condenser. The flask was shaken well and placed inside the microwave oven and irradiated (monomode, 210 W, 150° C.) for 6-9 minutes in parts. After completion the reaction was worked up as in example I and provided a viscous liquid; 51% yield; ¹H NMR (CDCl₃) δ 7.16 (1H, t, J=8.07 Hz), 6.98 (3H, m), 6.83 (1H, d, J=16.15 Hz), 6.70 (3H, m), 5.21 (1H, brd), 3.84 (6H, s); ¹³C NMR (CDCl₃) δ 156.1, 147.2, 139.1, 134.7, 129.8, 129.1, 128.9, 126.5, 119.0, 114.6, 112.8, 103.4 and 56.3.

### Example VIII

### Synthesis of 3,4-Dihydroxy-3′-Methoxy Stilbene

### From Formula I Where R₂ And R₃=OH, R₇=OCH₃, R₁, R₄, R₅, R₆, R₈, R₉, R₁₀=H

A mixture of 3,4-dihydroxybenzaldehyde (0.0181 mol), 3-methoxyphenylacetic acid (0.0151 mol), sodium acetate (4 g) and methyl imidazole (2 ml) were taken in a 100 ml round bottom flask fitted with a condenser. The flask was shaken well and placed inside the microwave oven and irradiated (monomode, 210 W, 170° C.) for 6-9 minutes in parts. After completion the reaction was worked up as in example I and provided viscous product; 61% yield ¹H NMR (CDCl₃) δ 7.22 (1H, t, J=8.07 Hz), 7.01 (2H, m), 6.94 (4H, m), 6.79 (2H, m), 6.70 (3H, m), 4.88 (2H, brd), 3.78 (3H, s); ¹³C NMR (CDCl₃) δ 159.8, 143.7, 143.5, 139.0, 130.9, 129.6, 128.5, 126.9, 120.2, 119.1, 115.5, 113.0, 111.6 and 55.3.

### Example IX

### Synthesis of 3,4′,5-Trihydroxy Stilbene (Resveratrol)

### From Formula I Where R₂ And R₃=OH, R₇=OCH₃, R₁, R₄, R₅, R₆, R₈, R₉, R₁₀=H

A mixture of 3,5-diacetyloxybenzaldehyde (0.0112 mol), 4-hydroxyphenylacetic acid (0.0135 mol), piperidine (3 ml) and methyl imidazole (3 ml) were taken in a 100 ml round bottom flask fitted with a condenser. The flask was shaken well and placed inside the microwave oven and irradiated (monomode, 210 W, 150° C.) for 6-9 minutes in parts. After completion the reaction was worked up as in example I and provided viscous solid; 66% yield ¹H NMR (CD₃COCD₃) δ 7.66 (2H, d, J=8.48 Hz), 7.30 (4H, m), 7.15 (2H, d, J=8.48 Hz), 6.90 (1H, s), 2.29 (6H, s). Further acetylated stilbene is treated with sodium methoxide in anhydrous tetrahydro furan and methanol to obtain resveratrol as a solid compound. ¹H NMR (CD₃COCD₃) δ 7.40 (2H, d, J=8. 07 Hz), 7.01 (4H, m), 6.53 (2H, s), 6.25 (1H, s); ¹³C NMR (CD₃COCD₃) δ 159.2, 158.3, 140.1, 130.1, 129.1, 128.8, 126.7, 116.3, 105.1 and 101.9. Resveratrol can also be directly obtained in the above reaction by taking 3,5-dihydroxybenzaldehyde in place of acetyloxybenzaldehyde.

### Example X

### Synthesis of 4-Hydroxy-3,3′,4′,5′-Tetramethoxy Stilbene (Combretastatin Analog)

### From Formula I Where R₃=OH, R₂, R₇, R₈ And R₉=OCH₃, R₁, R₄, R₅, R₆, R₁₀=H

A mixture of 4-hydroxy-3-methoxybenzaldehyde (0.0164 mol), 3,4,5-trimethoxyphenylacetic acid (0.0246 mol), triethylammine (5 ml) and methyl imidazole (3 ml) were taken in a 100 ml round bottom flask fitted with a condenser. The flask was shaken well and placed inside the microwave oven and irradiated (monomode, 210 W, 170° C.) for 6-9 minutes in parts. After completion the reaction was worked up as in example I and provided viscous product; 54% yield ¹H NMR (CDCl₃) δ 6.98 (1H, s), 6.85 (1H, d, J=8.48 Hz), 6.77 (1H, d, J=8.48 Hz), 6.51 (2H, m), 5.54 (1H, s), 3.90 (3H, s), 3.87 (3H, s), 3.76 (6H, s).

### Example XI

### Synthesis of 4-Hydroxy-3,4′-Dimethoxy Stilbene (By Conventional Method)

### From Formula I Where R₃=OH, R₄ And R₈=OCH₃, R₁, R₂, R₅, R₆, R₇, R₉, R₁₀=H

A mixture of 4-hydroxy-3-methoxybenzaldehyde (0.0164 mol), 4-methoxyphenylacetic acid (3.01 g, 0.0181 mol), piperidine (5 ml), acetic acid (10 ml) and dimethylformamide were taken in a round bottom flask and the reaction mixture was refluxed for 5-6 hours on heating mental. The cooled mixture was poured into ice-cold water and extracted with ethyl acetate. The organic layer was washed with water, brine and then organic layer dried over sodium sulphate. The solvent was evaporated under reduced pressure to obtain liquid which was purified on silica gel by column chromatography, 8:2 mixture of hexane and ethyl acetate provided stilbene, white solid; 37% yield (spectral data as in example I) and 6:4 mixture of hexane and ethyl acetate provided α-phenyl cinnamic acid as solid. NMR data of a-phenyl cinnamic acid; ¹H NMR (DMSO-D₆) δ 7.51 (1H, s), 6.93 (2H, d, J=8.48 Hz), 6.87 (3H, m), 6.55 (1H, d, J=8.48 Hz), 6.34 (1H, s), 3.55 (3H, s), 3.17 (3H, s).

### Example XII

### Synthesis of 1-(4′-Hydroxy-3-Methoxy)Phenyl-2-(1′-Naphthyl)Ethene

### From Formula I Where R₈=OH, R₁+R₂=phenyl, R₃, R₄, R₅, R₆, R₇, R₉, R₁₀=H

A mixture of 1-naphthaldehyde (0.0160 mol), 4-hydroxy-3-methoxyphenylacetic acid (0.0181 mol), ammoniumacetate (0.0246 mol) and diethylene glycol (3 ml) were taken in a 100 ml round bottom flask fitted with a condenser. The flask was shaken well and placed inside the microwave oven and irradiated (monomode, 250 W, 190° C.) for 8 minutes in parts. After completion the reaction was worked up as in example I and provided viscous liquid; 53% yield; solid, (m.p. 83-86° C.); ¹H NMR (CDCl₃) δ 8.22 (1H, d, J=7.68 Hz), 7.85 (1H, d, J=8.23 Hz), 7.77 (3H, m), 7.51 (3H, m), 7.11 (2H, m), 7.08 (IH, d, J=17.05 Hz), 6.95 (1H, d, J=8.23 Hz), 5.74 (1H, s), 3.91 (3H, s); ¹³C NMR (CDCl₃) δ 146.8, 145.8, 135.3, 133.8, 131.7, 131.4, 128.7, 127.8, 126.0, 125.8, 123.8, 123.6, 123.0, 120.6, 114.7, 108.6 and 56.0. HREIMS data: m/z [M+H]⁺ for C₁₉H₁₇O₂, calculated =277.3440; observed =277.3441.

### THE MAIN ADVANTAGES OF THE PRESENT INVENTION

The main advantage of the present invention is to provide a process to prepare high valued pharmacologically important 2- or 4-hydroxy substituted stilbenes from substituted arylaldehyde and substituted aryl acetic acid with at least one hydroxy substituent at 2- or 4-position of either arylaldehyde or aryl acetic acid.

A process, to employ ecofriendly microwave technique for the preparation of substituted 2- or 4-hydroxystilbenes.
1. A process to prepare 2- or 4-hydroxy substituted stilbenes in much shorter reaction time.
2. A process to prepare 2- or 4-hydroxy substituted stilbenes in good yield (37-66%).
3. A process for the preparation of 2- or 4-hydroxy substituted stilbenes in high purity with minimum or no side products such as cinnamic acid and polymerized product.
4. A process to develop a microwave-assisted preparation of 2- or 4-hydroxy substituted stilbenes where both condensation and decarboxylation unexpectedly occurred in one step.
5. A process to prepare 2- or 4-hydroxy substituted stilbenes in one pot.
6. A process wherein the ionic liquids used as solvent are recyclable.
7. A process for easy workup as well as purification of the product.
8. A process which utilizes less or non hazardous chemicals.
9. A process which requires cheaper chemical reagents.
10. A process to develop industrially viable process towards formation of high valued at 2- or 4-hydroxy substituted stilbenes.
11. An economical and industrial viable process for the preparation of high valued 2- or 4-hydroxy substituted stilbenes.

What is claimed is:

1. A single step microwave induced process for the preparation of substituted stilbenes and its analogs of general formula I
wherein, at least one substituent being OH amongst R₁, R₃, R₅, R₆, R₈, R₁₀, and rest of substituents amongst R₁ to R₁₀, being H or OH or OCH₃ or CH₃COO or halogen or nitro or combinations thereof, the said process comprising steps of: 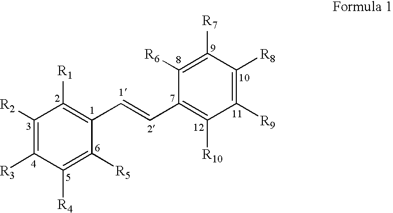
a) reacting substituted arylaldehyde and substituted aryl acetic acid with at least one hydroxy substituent at 2- or 4-position of either arylaldehyde or aryl acetic acid in the presence of a base, and/or an acid and a solvent by refluxing under conventional or microwave irradiation for a period ranging between 1 min-16 hrs,
b) transferring the reaction mixture of step (a) and washing the residue with an organic solvent,
c) washing the organic solution of step (b) with aqueous sodium bicarbonate, brine and water,
d) drying the organic layer of step (c) over anhydrous sodium sulphate, filtering and evaporating to dryness to completely remove the solvent to obtain a residue,
e) purifying the residue of step (d) by known methods to obtain the required substituted 2- or 4-hydroxy stilbenes of general formula (I).

2. A process according to claim 1, wherein the process for the preparation of 2- or 4-hydroxy substituted stilbenes is carried out in one step without any addition of decarboxylating agent.

3. A process according to claim 1, wherein the presence of at least one hydroxy substituent at 2- or 4-position of either arylaldehyde or aryl acetic acid is required for condensation-decarboxylation to occur in one step.

4. A process according to claim 1, wherein the base used is selected from the group consisting of collidine, triethylamine, pyridine, piperidine, sodium acetate, ammonium acetate, imidazole, methyl imidazoles and the combination thereof.

5. A process according to claim 1, wherein acid used is selected from the group consisting of formic acid, acetic acid, propionic acid and the combination thereof.

6. A process according to claim 1, wherein solvent used is selected from the group consisting of ethylacetate, dimethylformamide, ethanediol, diethylene glycol, dimethoxyethylene glycol, dimethyl sulphoxide, ionic liquids and the combination thereof.

7. A process according to claim 1, wherein the base used serve the dual purpose of a base as well as of a solvent.

8. A process according to claim 1, wherein developed process used is applied equally successfully on aromatic ring in aryalaldehydes and arylacetic acids other than benzaldehydes and phenylacetic acids such as naphthyl, phenanthryl, pyridyl, indyl, furyl, thiazolyl ring.

9. A process according to claim 1, wherein microwave enhances the yield of product stilbenes as compared to conventional method by 30-40%.

10. A process according to claim 1, wherein the claimed process is found workable in both a monomode and a multimode microwave.

11. A process according to claim 1, wherein the microwave irradiation frequency used is in the range of 900 to 3000 MHz, more preferably in the range of 2450 to 2455 MHz.

12. A process according to claim 1, wherein the reaction is performed in a monomode microwave organic synthesizer operated at 50 W-300 W power level with 100-250° C. for 1-20 min.

13. A process according to claim 1, wherein the temperature attained in case of monomode microwave is ranging from 100-250° C., preferably between 120-190° C.

14. A process according to claim 1, wherein the reaction is carried out in a domestic microwave oven operated at 700 W-1500 W power level for 1 min-30 min.

15. A process according to claim 1, wherein the product is formed by refluxing substrates by thermal heating for a period ranging between 2-16 hrs preferably, between 2-6hrs.

16. A process according to claim 1, wherein the mole ratio between substituted arylaldehyde and arylacetic acids is ranging between 1:1 to 1:4 moles.

17. A process according to claim 1, wherein the mole ratio between the substituted arylaldehyde and the base is ranging between 1:1 to 1:10 moles preferably, being 1:3 moles.

18. A process according to claim 1, wherein the mole ratio between the substituted arylaldehyde and acid is ranging between 1:1 to 1:20 moles preferably, being 1:10 moles.

19. A process according to claim 1, wherein developed process provides 2- or 4-hydroxy substituted stilbenes in high purity with no or minimum side products.

20. A process according to claim 1, wherein claimed process can be used for the preparation of large number of substituted stilbenes, ethenes and analogs by taking different substrate.
